(19) United States
(12) Patent Application Publication
(10) Pub. No.: US 2025/0194960 A1
(43) Pub. Date: Jun. 19, 2025

(54) METHOD FOR ADAPTING A HEARING DEVICE, HEARING DEVICE AND DATA CARRIER

(71) Applicant: Sivantos Pte. Ltd., Singapore (SG)

(72) Inventors: Rosa-Linde FISCHER, Erlangen (DE); Tobias WURZBACHER, Erlangen (DE)

(21) Appl. No.: 19/069,491

(22) Filed: Mar. 4, 2025

Related U.S. Application Data
(63) Continuation of application No. PCT/EP2023/062799, filed on May 12, 2023.

(30) Foreign Application Priority Data
Sep. 8, 2022 (DE) 10 2022 209 383.6

(51) Int. Cl. A61B 5/12; A61B 5/00; H04R 25/00

(52) U.S. Cl. CPC A61B 5/123; A61B 5/742; H04R 25/558; H04R 25/70; H04R 2225/55

(57) ABSTRACT

A method adapts a hearing device having at least one hearing aid, a movement sensor, and a display unit. At least one test measurement is carried out in which an acoustic test signal is generated. A movement sensor detects a movement of the hearing device user in response to the test signal as a test result, and visual feedback is generated on the display unit on the basis of the test result.

### CROSS-REFERENCE TO RELATED APPLICATION

This application is a continuation, under 35 U.S.C. § 120, of copending International Patent Application PCT/EP2023/062799, filed May 12, 2023, which designated the United States; this application also claims the priority, under 35 U.S.C. § 119, of German Patent Application DE 10 2022 209 383.6, filed Sep. 8, 2022; the prior applications are herewith incorporated by reference in their entirety.

### FIELD AND BACKGROUND OF THE INVENTION

The invention relates to a method for adapting a hearing device, in particular a hearing aid, as well as a use of the method, a hearing device, and software on a data carrier.

Hearing aids are wearable hearing devices (hearing aid devices), which are used to treat the hard-of-hearing or the hearing-impaired. To accommodate the numerous individual requirements, different structural forms of hearing devices such as behind-the-ear hearing aids (BTE) and hearing aids having an external receiver (RIC: receiver in the canal) as well as in-the-ear hearing aids (ITE), for example also concha hearing aids or channel hearing aids (ITE: in-the-ear, CIC: completely-in-channel, IIC: invisible-in-the-channel) are provided. The hearing devices listed as examples are worn on the outer ear or in the auditory canal of a hearing device user. In addition, however, bone vibrator hearing aids, implantable or vibrotactile hearing aids are also available on the market. In this case, the impaired hearing is stimulated either mechanically or electrically.

Such hearing devices or hearing aids have in principle as essential components an input transducer, an amplifier, and an output transducer. The input transducer is generally an acousto-electric transducer, such as a microphone, and/or an electromagnetic receiver, such as an induction coil or a (radio frequency, RF) antenna. The output transducer is usually implemented as an electro-acoustic transducer, for example, as a miniature loudspeaker (receiver), or as an electromechanical transducer, such as a bone vibrator receiver. The amplifier is typically integrated into a signal processing unit. The energy is typically supplied by a battery or a rechargeable accumulator.

The input signals recorded by the input transducers are typically multichannel, which means that the input signals are divided into multiple individual frequency channels, wherein each frequency channel covers a frequency band of a specific spectral width. For example, a hearing aid can in this case have 48 (frequency) channels in a frequency range between 0 KHz (kilohertz) and 24 kHz, wherein the individual signal components of the input signal can be individually processed, in particular individually filtered and/or amplified, in the channels by means of the signal processing unit.

An optimum adaptation to the requirements of a hearing device user can be achieved by a skilled setting of the time-dependent and frequency-dependent amplification of an acoustic input signal by a hearing device. The problem arises here of determining the most optimum possible time-dependent and frequency-dependent amplification for the hearing device user or hearing aid user/wearer.

The (initial) adaptation of a hearing aid is intended in particular to find the best amplification/sound and algorithm settings for all individual hearing aid wearers. The most important inputs are the audiometric data of the hearing aid wearer (bone conduction/air conduction threshold, degree of discomfort). In addition, some adaptation methods also take into consideration other information of the hearing aid wearer such as age, sex, and experience with hearing aids in order to further optimize the initial adaptation.

There are people, such as small children or people having cognitive impairments, who are not capable of reliably following the instructions for hearing threshold measurement. This means that the procedure of the verbal instruction or the keypress, as soon as a sinusoidal tone is just perceptible—if they were understood and can be followed at all—results in a broad scattering of the responses and test results. Therefore, only a low data quality of the hearing thresholds is available for the adaptation, which is the most important information for an appropriate hearing aid adaptation.

This problem is presently solved by simple behavior tests, in which various noises are used as test signals in order to stimulate specific frequency ranges, wherein the reaction of the child/person to the test signal is observed. To receive responses at all, a conditional learning method is performed before the approximation behavior in the perception of a noise.

If the child or person is capable of actively participating, understanding and following simple rules, conditioned game audiometry can also take place. In this case, the audiometry is introduced as a game in order to obtain the motivation for reactions.

In addition, there are tablet-based solutions, in which various frequencies are represented, for example, by different animals. The child or person is to click on the respective animal in this case as soon as they hear the corresponding sound. Such solutions increase the level of motivation, but still presume the capability of understanding the instructions and following them reliably.

### SUMMARY OF THE INVENTION

The invention is based on the object of specifying a particularly suitable method for adapting a hearing device. The invention is furthermore based on the object of specifying a particularly suitable use of the method, a particularly suitable hearing device, and particularly suitable software on a data carrier.

The object is achieved according to the invention with respect to the method by the features of the independent method claim and with respect to the use by the features of the independent method of use claim and with respect to the hearing device by the features of the independent hearing device claim and with respect to the software by the features of the independent software based claim. The advantages and embodiments listed with regard to the method are also transferable accordingly to the use and/or the hearing device and/or the software and vice versa.

The conjunction “and/or” is to be understood here and hereinafter to mean that the features linked by means of this conjunction can be formed both jointly and also as alternatives to one another.

Insofar as method steps are described hereinafter, advantageous embodiments for the hearing device result in particular in that it is designed to carry out one or more of these method steps.

With the foregoing and other objects in view there is provided, in accordance with the invention, a method for adapting a hearing system having at least one hearing aid with a movement sensor and a display unit. The method includes carrying out at least one test measurement, which contains the sub-steps of: generating an acoustic test signal, detecting a movement of a hearing device user in reaction to the acoustic test signal using the movement sensor resulting in a test result, and generating visual feedback on the display unit depending on the test result. A signal processor of the hearing aid is set in dependence on the test result.

The method according to the invention is intended, and also suitable and configured, for adapting a hearing device, in particular a hearing aid device. The hearing device contains at least one hearing aid (HA) and a movement sensor as well as a display unit.

The hearing device is used in particular to treat a hearing-impaired user (hearing device user). The hearing aid is configured here to record sound signals from the surroundings and output them to the hearing device user. For this purpose, the hearing device contains at least one input transducer, in particular an acousto-electric transducer, such as a microphone.

The input transducer records sound signals (noises, sounds, speech, etc.) from the surroundings in operation of the hearing device or the hearing aid and converts each of them into an electrical input signal. The input signal is in particular embodied as multichannel in this case. In other words, the acoustic signals are converted into a multichannel input signal. The input signal thus contains multiple frequency channels, in particular at least two, preferably at least 20, particularly preferably at least 40, for example 48, (frequency) channels, each of which covers an assigned frequency band of a frequency range of the hearing device. For example, a frequency range between 0 KHz and 24 kHz is divided here into 48 channels so that input signals having 48channels are generated.

The hearing aid furthermore comprises an output transducer, in particular an electro-acoustic transducer, such as a receiver or miniature loudspeaker. An electrical (multichannel) output signal is generated from the electrical (multichannel) input signal in that the input signal, or the individual frequency or signal channels, are processed and modified (e.g., amplified, filtered, damped) in a signal processing unit. The setting of the signal processing unit, in particular with regard to the signal amplification, takes place according to the invention in the course of the adaptation on the basis of a test result of a test measurement.

According to the method, at least one test measurement is carried out in which an acoustic (test) signal (test stimulus, test sound, test stimuli) is generated. By means of the movement sensor, a movement of the hearing device user is detected as a reaction to the signal (or an absence of such a movement) as a test result, wherein visual feedback is generated on the display unit depending on the test result.

According to the method, a signal amplification or signal processing of the hearing aid, thus the signal processing unit, is set on the basis of the test result. In other words, the hearing aid settings or hearing aid parameters are changed depending on the test measurement or the test result. The hearing aid device or the hearing aid is therefore set or adapted on the basis of the test results or specific hearing thresholds. A particularly suitable method for adapting the hearing device is thus implemented, which is particularly suitable for small children or people having cognitive impairments due to the visual feedback.

In an advantageous refinement, multiple test measurements, thus at least two test measurements, are carried out in succession. A more reliable adaptation of the hearing device is thus ensured.

In a suitable embodiment, a movement analysis of the detected movement sensor data is carried out to determine the hearing threshold, wherein a signal level of the test signal is changed (increased/reduced) depending on the movement analysis. In particular the signal level of a following test measurement is thus changed, so that successive test measurements build on the preceding test results, and therefore implement a successively more accurate determination of the hearing threshold.

Multiple test measurements are expediently carried out, and therefore a measure is determined for the hearing thresholds of the hearing device user. The evaluation of the test results can be carried out by a person/tester, such as an audiologist, or by software or artificial intelligence, for example a neural network.

A hearing threshold method is thus implemented which offers the possibility of an automated, closed hearing threshold test. The reaction of the hearing device user is objectified by movement sensor data which are combined with the knowledge about the presented test signals/test sounds. In addition, the hearing device user receives visual feedback about his behavior for motivation. Visual feedback gives the tester objective feedback about the test performance.

Presently, the reaction of a hearing threshold test by the subjective interpretation of the behavior of a test person is assessed by the tester. The proposed technical solution of the method according to the invention supplies objective feedback about the reaction to a perceived test signal on the basis of movement sensor data. The time for training the response behavior is thus reduced, the reliability of the measurement is increased, and the motivation for this measurement is increased. The method is not only suitable, but is particularly suitable for people having restricted comprehension of the test sequence, for example very small children or cognitively impaired people. In addition, these people could benefit from a method with a clear reward so that they remain motivated for the measurement protocol.

The data of a movement sensor, which is integrated either in the hearing aid, other technical devices, or independent movement sensors, are combined with the knowledge about the presentation properties of the test stimulus via the hearing aid or other technical devices. In the background, for example, an algorithm analyzes the presentation time, volume, and frequency of the test sounds and derives the hearing threshold for multiple frequencies or frequency channels of the hearing aid from the hit and miss rate of the response behavior detected by the movement sensor.

In one conceivable embodiment, the signal level of the test signal is changed such that a difference between the test signal having the lowest perceptible signal level and the test signal having the highest perceptible signal level reaches or falls below a predetermined threshold value. In other words, the steps for hearing threshold determination (movement analyses, level reduction or increase) are repeated until the difference between the test signal having the softest audible level and the test signal having the strongest non-audible level is below a predetermined (for example parameterizable) threshold. The threshold values can be, for example, less than 6 dB, 3 dB, or 1 dB. The hearing threshold of the hearing device user is then between these signal levels.

In an expedient design, the test signal is output by the hearing aid. In other words, the hearing aid generates the test signal. For this purpose, the hearing aid comprises, for example, a signal generator, on which an electrical test signal is generated that is output as an acoustic test signal by the output transducer.

The advantage of an implementation in the hearing aid in relation to external loud speakers or headphones is bypassing the need for calibration. Due to the possibility of muting the microphone or input transducer, moreover fewer external noises interfere with the test sequence. The hearing aid implementation would furthermore offer the possibility of carrying out an in situ hearing threshold test or a check of the real ear amplification. In addition, an automation of the method could be used to collect hearing test data over a longer period of time and in daily life (for example at home). Typical modern hearing aids have installed movement sensors, which are a requirement for the proposed structure.

An additional or further aspect of the invention provides the use of the above-described method as a gamified hearing threshold test (hearing test). In particular gamified visual feedback is generated or used in this case. A particularly suitable application with regard to the determination of hearing thresholds in small children or people having cognitive impairments is thus implemented.

“Gamified” or “gamification” is to be understood as an application of game-typical elements in a game-external context. In this case, game design principles, game design concepts, and game mechanics are transferred to game-external applications and processes in order to solve problems and engage participants. The goal is increasing motivation of the user, interacting in an intensified manner with an application which is otherwise perceived as unchallenging, excessively monotonous, or excessively complex, or adopting desired behaviors.

For the implementation of a gamified hearing test with detection of objective behavior data by movement sensors, for example, three approaches are conceivable, which are also referred to hereinafter as run & jump, sprint competition, and dancing game.

The run & jump approach is an implicit method which is based on the natural evasion behavior: a manikin (game character) runs across the display screen. The test person is instructed to jump and shake their head as soon as an obstacle approaches from any direction.v Preceding test sounds indicate, for example, whether the next obstacle comes from the left or right. The reaction of the test person to the approaching obstacle is faster if the test sound is perceived. The analysis algorithm calculates the hit and miss rate of the behavior reaction by the comparison of the auditive representation to the solely visual representation of obstacles. The visual feedback to the test subject—the manikin on the display screen reflects the behavior of the test subject, for example jumps or direction changes—is used to maintain the motivation of the test subject.

In the sprint competition approach, the instruction is given to start a “100 m” sprint (or ride, car/Formula One race, etc.) as soon as the test signal is perceived. The sounds of the test signal are varied in frequency and volume and presented in unpredictable time patterns (random stimulus intervals). An algorithm calculates the hearing threshold by the comparison of correct and incorrect start, which is indicated by the feedback of the movement sensor upon the “race start”. Visual feedback is used to maintain the motivation of the test person.

The “dancing game” approach is an implicit method which makes use of the intrinsic motivation to adapt to a perceived musical rhythm. The test stimuli are sound sequences which are defined by a specific pitch and volume. These sets are played back in a predefined periodicity (rhythm; beats per minute; optionally embedded in a song). An algorithm analyzes the correlation between the rhythmic movement of the test subject and the presented rhythm. Their correspondence indicates the perception of a specific sound sequence. The hearing threshold (level) for a specific frequency (pitch) is derived from the information about pitch and level of a set. The visual feedback to the test subject is used to maintain motivation. In addition, the parallel representation of the rhythmic behavior of the test person and the presented rhythm can be used to give direct objective feedback to the tester.

In general, all gamified hearing loss assessments could also be carried out by untrained people, for example by the parents or caregiver of a hearing-impaired person. For this purpose, an app implementation, for example for a smart phone or for a tablet computer, could be provided which is capable of putting the hearing aid in a measuring mode in order to present test sounds and collect movement sensor data. This is advantageous in particular with regard to an application in freely-purchasable or prescription-free hearing aids, so-called OTC hearing aids (over-the-counter, OTC). In the case of OTC hearing aids, the hearing aid wearer himself is responsible for configuring the hearing aid, including adapting and setting the sound. The hearing aid wearer therefore generally has to effectuate an initial adaptation without audiometric input data and without professional help. The self-adaptation (self-fitting, SF) is facilitated by the method.

The hearing device according to the invention contains a hearing aid, in particular an OTC hearing aid. The hearing device furthermore contains a movement sensor for detecting a (body) movement of the hearing device user and a display unit for displaying visual feedback as well as a controller, thus a control unit, for carrying out the above-described method.

The controller is generally configured—by programming and/or circuitry—in this case for carrying out the above-described method according to the invention. The controller is therefore specifically configured to carry out a test measurement, evaluate the detected movement data of the movement sensor, and as a result set or adapt the hearing aid.

In one preferred embodiment, the controller is formed at least at the core by a microcontroller having a processor and a data memory in which the functionality for carrying out the method according to the invention is implemented by programming in the form of operating software (firmware), so that the method—possibly in interaction with a device user—is carried out automatically upon execution of the operating software in the microcontroller. The controller can alternatively also be formed in the scope of the invention, however, by a non-programmable electronic component, such as an application-specific integrated circuit (ASIC), or by an FPGA (field-programmable gate array), in which the functionality for carrying out the method according to the invention is implemented using circuitry means.

In one suitable embodiment, the display unit and the controller are embodied as an operating and display device coupled for signaling with the hearing aid. The in particular mobile operating and display device is, for example, a mobile telephone, in particular a mobile telephone having a computer function or a smart phone or also a tablet computer.

The operating and display device comprises for this purpose, for example, saved application software (operating software), by means of which the audio and/or video material is played back, and by means of which the test results of the hearing device user are detected. On the basis of the test results, the application software subsequently sets the settings or amplification of the hearing aid. The application software is preferably installable or installed for this purpose as a so-called app or mobile app (mobile application, smart phone app) on the operating and display device.

This embodiment proceeds here from the consideration that modern operating and display devices, such as smart phones or tablet computers in particular, are widespread in today's society and are generally available and accessible at any time to a user. In particular, the user of the hearing aid device has with high probability essentially such an operating and display device in his household.

The surfaces of smart phones or tablet computers, which are typically configured as touchscreens (display, display screen) furthermore permit particularly easy and intuitive operation of the application software of the operating and display device thus formed. A smart phone or tablet computer can thus be retrofitted particularly cost-effectively for the adaptation of the hearing aid device.

The operating and display device comprises an internal controller, which is formed at least at the core by a microcontroller having a processor and a data memory in which the functionality for carrying out the method is implemented by programming in the form of application software, so that the method or the determination of the operating state of the hearing aids—possibly in interaction with the user—is carried out automatically upon execution of the application software in the controller.

An additional or further aspect of the invention provides software on a medium or data carrier for carrying out or executing the above-described method. This means that the software is stored on a data carrier and is intended, and also suitable and designed, for carrying out the above-described method. Particularly suitable software for the adaptation of a hearing device is thus implemented, using which the functionality for carrying out the method according to the invention is implemented by programming. The software is therefore in particular operating software (firmware), wherein the data carrier is, for example, a data memory of the controller.

Other features which are considered as characteristic for the invention are set forth in the appended claims.

Although the invention is illustrated and described herein as embodied in a method for adapting a hearing device, a hearing device and a data carrier, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

FIG. 1 is a block diagram showing a method according to the invention;

FIG. 2 is an illustration of a hearing device having a hearing aid and having an auxiliary device; and

FIG. 3 is an illustration of three rhythm-volume diagrams.

### DETAILED DESCRIPTION OF THE INVENTION

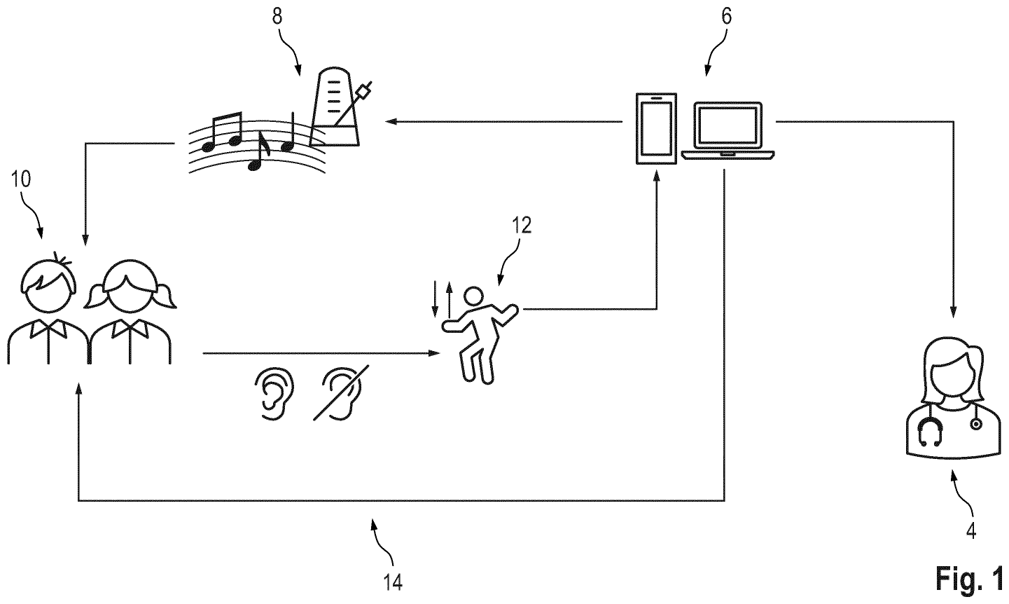

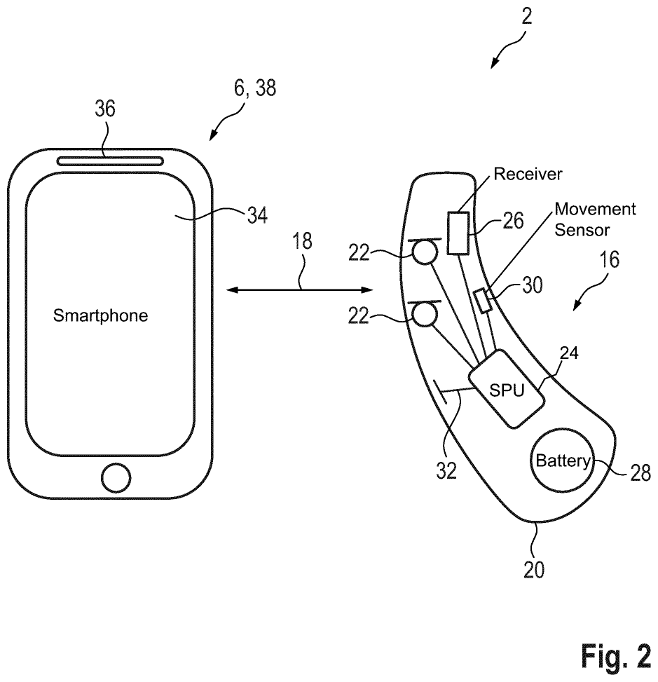

Referring now to the figures of the drawings in detail and first, particularly to FIGS. 1 and 2 thereof, there is shown an overview of a first embodiment of a method according to the invention for adapting a hearing device 2 (FIG. 2). FIG. 1 shows in particular a sequence of a gamified hearing threshold method in this case. The method is carried out in this case, for example, by a tester 4 (investigator, test coordinator).

At least one test measurement is carried out by means of a display and operating device 6, in which an acoustic (test) signal (test stimulus, test sound, test stimuli) 8 is generated. The signal 8 can be embodied as one or more sounds, a rhythm, a sweep, or the like. The display and operating device 6 can be embodied, for example, as a computer or as an app on a smart phone or tablet.

A hearing device user (test person, test subject) 10, for example a child, is subjected to the signal 8 where a body movement of the subject is now tracked, wherein a distinction is made in particular between movements which take place with the signal 8 and those which do not take place with the signal 8. The reaction is objectified by movement sensor data 12 which are combined with the knowledge about the presented test signals/test sounds. The display and operating device 6 analyzes the detected (body) movement with respect to a correlation of the signal 8 and the movements and as a result adapts the stimuli or parameters (volume, frequency, beats/minute, etc.) of the signal 8. In addition, the hearing device user 10 receives visual feedback 14 about his behavior from the display and operating device 6 for motivation. The hearing device user 10 can optionally additionally also receive acoustic feedback by sounds or the like. The visual feedback 14 gives the tester (investigator, test coordinator) 4 objective feedback about the test performance. The hearing aid device 2 is set or adapted on the basis of the test results or determined hearing thresholds.

FIG. 2 shows a simplified and schematic representation of the fundamental structure of the hearing device 2.

The hearing device 2 is embodied in particular as a hearing aid device in the form of a hearing system having a hearing aid 16 and an external auxiliary device in the form of the display and operating device 6. The hearing aid 16 is configured in this case, for example, as a behind-the-ear hearing aid device (BTE). The hearing aid 16 can in particular be an OTC hearing aid. The hearing aid 16 and the display and operating device 6 are coupled with one another for signaling by means of a wireless communication connection 18. The communication connection 18 is preferably embodied as a radio connection, for example as a Bluetooth or RFID connection.

The hearing aid 16 contains, as schematically shown in FIG. 2, a device housing 20, in which one or more microphones 22, also referred to as acousto-electric (input) transducers, are installed. The sound or the acoustic signals in the surroundings are recorded using the microphones 22 and converted into an electrical audio signal.

The audio signal is processed by a signal processing unit 24, which is also arranged in the device housing 20. On the basis of the audio signal, the signal processing unit 24 generates an output signal, which is conducted to a loudspeaker or receiver 26. The receiver 26 is embodied in this case as an electro-acoustic (output) transducer, which converts the electrical output signal into an acoustic signal and outputs it. In the BTE hearing aid 16, the acoustic signal is possibly transmitted via a sound tube or external receiver (not shown), which to an earmold seated in the auditory canal, to the eardrum of a hearing aid device user. However, an electro-mechanical transducer is also conceivable as the receiver 26, such as in the case of a bone vibration receiver.

The power supply of the hearing aid 16 and in particular the signal processing unit 24 is performed by means of a battery 28 accommodated in the device housing 20.

The signal processing unit 24 is coupled for signaling to a movement sensor 30 of the hearing aid 16. The movement sensor 30 is intended and configured for detecting movements of the hearing device user 10. The movement sensor 30 is intended and configured to detect three-dimensional movements, in particular translational and/or rotational movements. The movement sensor 30 is designed in this case, for example, as an accelerometer and/or as a gyroscope, thus as a gyroscopic (location) sensor. The movement sensor 30 can alternatively also be a light sensor for detecting light signals in the surroundings of the hearing aid 16, or a pulse or blood pressure sensor for detecting pulse or blood pressure changes of the hearing device user 10. A movement sensor 30 which comprises a combination of accelerometer and/or gyroscope and/or pulse sensor and/or blood pressure sensor and/or light sensor is also possible.

The signal processing unit 24 is furthermore led for signaling to a transceiver 32 of the hearing aid 16. The transceiver 32 is used to transmit and receive wireless signals by means of the communication connection 18. The transceiver 32 can be embodied in this case, for example, as an induction coil.

In the exemplary embodiment of FIG. 2, a separate mobile operating and display device 6 is coupled for signaling with the hearing aid 16 by means of the communication connection 18. The schematically shown operating and display device 6 is in particular a smart phone. The smart phone 6 comprises a touch-sensitive display unit (display) 34, which is also designated hereinafter as a touch screen. The smart phone 6 furthermore comprises at least one loudspeaker 36 for emitting acoustic signals.

The signal coupling between the smart phone 6 and the transceiver 32 of the hearing aid 16 is produced here via a corresponding integrated transceiver (not described in more detail), for example a wireless or radio antenna, of the smart phone 6.

The smart phone 6 contains an integrated controller, which is substantially formed by a microcontroller having implemented application software 38. The application software 38 is preferably a mobile app or a smart phone app which is stored in a data memory of the controller. The controller represents the application software 38 on the touch screen 34 in operation, wherein the application software 38 is operable by means of the touch-sensitive surface of the touch screen 34 by a hearing device user 10.

A second exemplary embodiment of a method according to the invention for adapting the hearing aid device 2 to the hearing requirements of the hearing device user 10 is explained hereinafter.

The core concept of the method is a movement sensor in the hearing aid 16 (preferred, but can also be integrated in an auxiliary device such as the smart phone 6), by means of which a rhythm or beat (or the changes of the former) of the reaction of a test person 10 to various test signals or sounds (volume & frequency) are detected. The test signals are those rendered by an internal test signal or sound generator (receiver 26) of the hearing aid 16 (preferably, but an auxiliary device for sound generation can alternatively also be provided). According to the method, inferences about the hearing threshold of the test person 10 are drawn from the test signals and the detected body movements. A visual representation of the ego movement in real time (with a bouncing dot or a computer character) on the display unit or the touch display 24 increases the level of willingness and attentiveness for the test.

Children and young people are a target group of the application, who are easier to motivate for such a movement-based audiometry in comparison to classic audiometry.

The method explained hereinafter is also suitable for the OTC/SF case for young people or adults in order to determine the hearing threshold. In this case, the test person 10 is also the test coordinator 4. For this purpose, the hearing aid 16 has integrated movement sensor 30 and a sound generator 26 as well as a smart phone/tablet as an operating and display device 6 having a wireless connection 18 to the hearing aid 16 is used as the hearing device 2. In one embodiment, a professional test coordinator (such as a hearing aid audiologist, etc.) could also be connected remotely via the application software 38, or only as needed in case of a failure.

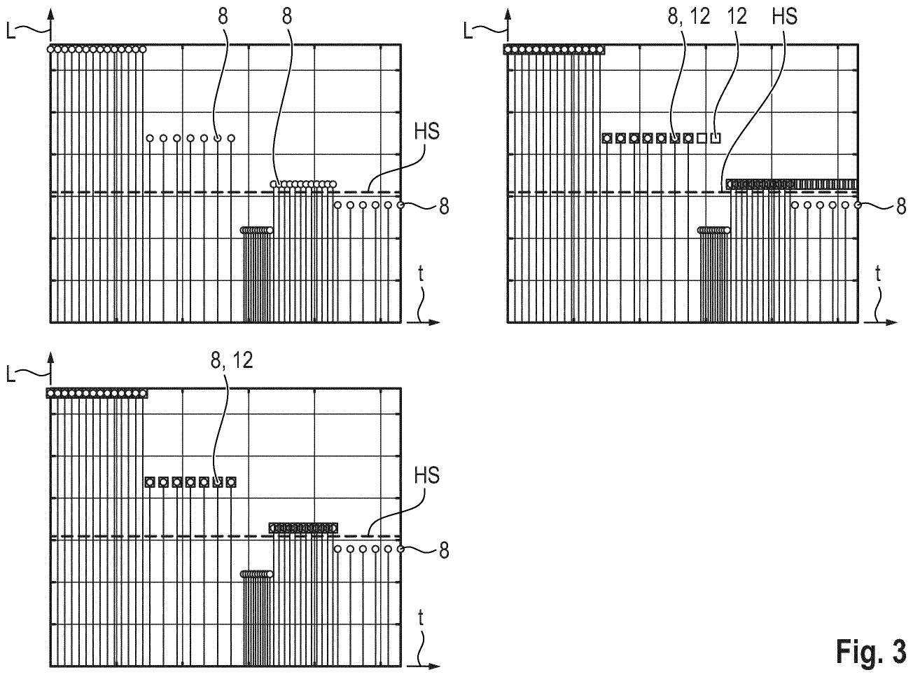

The test person 10 wears the hearing aid 16, which is connected to the smart phone/tablet 6. The tester 4 starts the movement audiometry via the application software 38. The application software 38 parameterizes the sound generator 26 in the hearing aid 16 so that it plays back sounds in a rhythmic manner. The rhythm itself changes with time in an unpredictable manner. Together with the rhythm change, the volume of the sound also changes. This is shown, for example, in FIG. 3.

FIG. 3 shows sets of test sounds which represent combinations of levels and rhythmic patterns. Three rhythm-volume diagrams are shown in FIG. 3, wherein time t is plotted horizontally, thus along the abscissa axis (X axis) in each case and a volume L, for example in decibels (dB) is plotted along the vertical ordinate axis (Y axis) in each case. The test signals or test stimuli 8 are shown as circles having reference lines in relation to the time axis, the movement sensor data 12 as rectangles, and the hearing threshold HS as a horizontal dashed line. In FIG. 3, the test stimuli 8 and movement sensor data 12 are provided with reference signs solely by way of example.

In the top right and bottom left illustration of FIG. 3, movement sensor data (motion reaction) 12 about the movement of the test person, who shows the same rhythm as the audible set, are shown. The hearing threshold HS can be derived by variation of the level of the set. Test stimuli 8 which are correlated with corresponding movement sensor data 12 are classified as audible or perceptible, thus as above the hearing threshold HS, wherein test stimuli 8 without assigned or assignable movement sensor data 12 are classified as non-audible or non-perceptible, thus as below the hearing threshold HS. By varying the volume L of the test stimuli 8 and evaluating the movement sensor data 12, the location of the hearing threshold HS can therefore be successively determined.

The test person 10 is to execute movements in accordance with the sound playback. This can be a simple up-and-down or bobbing with the entire body or only the head. The movement sensor 30 tracks the movement of the test person 10 and transmits the data to the connected smart phone 6 or the application software 38 for real-time visualization of the ego movement on the display 34. The movement sensor data 12 are evaluated—especially the movement frequency—either in the application software 38 or already in the hearing aid 16.

It is not absolutely necessary (but is naturally helpful for the automatic analysis) for the movement and sounds to be synchronized. It is important that the movement frequency of the test person 10 changes with the sound stimuli. Optionally, a short training sequence can be carried out before the test in order to judge the capability of the test person 10 to react to the changes of the stimulus pattern with respect to regularity and synchronicity of the movement pattern and the reaction time: “How quickly does the test person react to changes”. These parameters could be used to match the test method and the analyses to the individual test person 10.

If a change of the movement pattern (for example, the frequency) can be established, it is concluded therefrom that the test person 10 can still hear the sound in the volume presented and the hearing threshold HS is not reached. The movement audiometry is continued and the level of the sound stimuli is reduced for the next rhythm change.

If the movement pattern (for example, the frequency) does not change or no movement can be established at all, the conclusion is that the test person 10 is not capable of hearing the sound in the presented volume and the sound has fallen below the hearing threshold HS. The movement audiometry is continued and the level of the sound stimulus is increased for the next rhythm change.

These steps (movement analyses, level reduction or increase) are repeated until the difference between the sound having the softest audible level and the sound having the strongest non-audible level is below a specific (parameterizable) threshold. The threshold values can be, for example, less than 6 dB, 3 dB, or 1 dB. The hearing threshold of the test subject is between these levels.

This procedure is to be repeated for various sound frequencies in order to determine the hearing threshold. The determined hearing threshold is stored for further use, for example for the (initial) adaptation and/or the fine tuning of the HA parameters.

The claimed invention is not restricted to the above-described exemplary embodiments. Rather, other variants of the invention can also be derived therefrom by a person skilled in the art in the context of the disclosed claims without departing from the subject matter of the claimed invention. In particular, all individual features described in conjunction with the various exemplary embodiments are furthermore also combinable in the scope of the disclosed claims in another manner without departing from the subject matter of the claimed invention.

The following is a summary list of reference numerals and the corresponding structure used in the above description of the invention:
2 hearing device
4 tester
6 operating and display device/smart phone
8 test signal
10 hearing device user
12 movement sensor data
14 feedback
16 hearing aid
18 communication connection
20 device housing
22 microphone
24 signal processing unit
26 receiver
28 battery
30 movement sensor
32 transceiver
34 display unit
36 loudspeaker
38 application software
t time
L volume
HS hearing threshold

1. A method for adapting a hearing system having at least one hearing aid with a movement sensor and a display unit, which comprises the steps of:
carrying out at least one test measurement, which comprises the sub-steps of:
generating an acoustic test signal;
detecting a movement of a hearing device user in reaction to the acoustic test signal using the movement sensor resulting in a test result; and
generating visual feedback on the display unit depending on the test result; and
setting a signal processor of the hearing aid in dependence on the test result.

2. The method according to claim 1, which further comprises carrying out a plurality of test measurements in succession.

3. The method according to claim 1, which further comprises determining, in a course of the at least one test measurement, a hearing threshold of the hearing device user for the acoustic test signal.

4. The method according to claim 3, which further comprises carrying out a movement analysis to determine a hearing threshold, wherein a signal level of the acoustic test signal is changed depending on the movement analysis.

5. The method according to claim 4, which further comprise changing the signal level of the acoustic test signal such that a difference between the acoustic test signal having a lowest perceptible signal level and the acoustic test signal having a highest perceptible signal level reaches or falls below a predetermined threshold value.

6. The method according to claim 1, which further comprises outputting the acoustic test signal via the hearing aid.

7. A method of performing a hearing threshold test for a hearing device user, which comprises the steps of:
performing the method according to claim 1; and
generating and using gamified visual feedback.

8. A hearing device, comprising:
at least one hearing aid;
a movement sensor;
a display unit; and
a controller for carrying out the method according to claim 1.

9. The hearing device according to claim 8, wherein said display unit and said controller are embodied as an operating and display device coupled for signaling with said at least one hearing aid.

10. A non-transitory data carrier storing computer executable instructions for carrying out the method according to claim 1 when the computer executable instructions are run on a computer.
